(19) United States
(12) Patent Application Publication
(10) Pub. No.: US 2017/0056206 A1
(43) Pub. Date: Mar. 2, 2017

(54) SYSTEMS AND DEVICES WITH FORCE ATTENUATING POLYMER GEL

(71) Applicant: Paul A. Glazer, Boston, MA (US)

(72) Inventor: Paul A. Glazer, Boston, MA (US)

(21) Appl. No.: 15/248,338

(22) Filed: Aug. 26, 2016

Related U.S. Application Data
(60) Provisional application No. 62/210,711, filed on Aug. 27, 2015.

(51) Int. Cl. A61F 2/46; A61F 13/04; A61F 2/44; A61F 2/80; A61F 5/01; A61F 5/055

(52) U.S. Cl. CPC A61F 2/4611; A61F 5/0125; A61F 5/055; A61F 2/4465; A61F 2/80; A61F 13/04; A61F 2002/4475; A61F 2002/4615; A61F 2002/4681; A61F 2005/563

(57) ABSTRACT

A device, e.g. a medical device, includes an energy absorbing polymeric compound. An orthopedic implant system includes an implant configured and adapted for implantation in orthopedic applications. An insertion tool is operatively connected to the implant during insertion of the implant. A hammer includes a layer of gel on a distal facing side configured to engage with the insertion tool during insertion of the implant to attenuate impact forces and protect the implant from damage.

### CROSS-REFERENCE TO RELATED APPLICATION

This application claims the benefit of priority under 35 U.S.C. §119(e) to U.S. Provisional Application No. 62/210,711, filed Aug. 27, 2015, which is incorporated herein by reference in its entirety.

### BACKGROUND OF THE INVENTION

1. Field of the Invention

This invention generally relates to polymeric compounds and in greater detail the invention relates to a viscoelastic, force-attenuating elastomeric compound and to its application in devices such as braces, implants, casting material, prosthetics, electronics cases and the like.

2. Description of Related Art

Devices such as braces, casts, cervical collars, prosthetics, implants, electronics cases, and the like can be subject to continuous static force and/or dynamic impact during use, and can subject the wearer or the object being held to continuous static force and/or dynamic impact. The respective injury, condition or device meant to be stabilized, remedied or protected by these respective devices can therefore experience additional trauma or discomfort due to the continuous static force and/or dynamic impact.

Such conventional methods and systems have generally been considered satisfactory for their intended purpose. However, there is still a need in the art for improved dynamic and/or static force attenuation. The present invention provides a solution for these problems.

### BRIEF DESCRIPTION OF THE DRAWINGS

So that those skilled in the art to which the subject invention appertains will readily understand how to make and use the devices and methods of the subject invention without undue experimentation, preferred embodiments thereof will be described in detail herein below with reference to certain figures, wherein:

FIG. 1 is perspective view of a partial cross-section of a knee brace constructed in accordance with embodiments of the present invention, showing a force-attenuating elastomeric compound embedded within the knee brace and at the interface between the knee brace and the user's skin;

FIG. 2 is perspective view of a partial cross-section of a mouth guard constructed in accordance with embodiments of the present invention, showing a force-attenuating elastomeric compound layered in the mouth guard;

FIG. 3 is cross-sectional perspective view of athletic tape constructed in accordance with embodiments of the present invention, showing the athletic tape having a force-attenuating elastomeric compound;

FIG. 4 is perspective view of a partial cross-section of an arm cast constructed in accordance with embodiments of the present invention, showing the arm cast having a force-attenuating elastomeric compound embedded between the cast lining and the hard cast exterior and outside of the hard cast exterior;

FIG. 5 is a cross-sectional side view of a cervical collar constructed in accordance with embodiments of the present invention, showing the cervical collar having a force-attenuating elastomeric compound at the mandibular, occipital and sternal support points;

FIG. 6 is perspective view of a schematic depiction of a mattress pad constructed in accordance with embodiments of the present invention, showing the mattress pad having a force-attenuating elastomeric compound at sacral and heel pressure points;

FIG. 7 is perspective view of a partial cross-section of a prosthetic sock and prosthesis constructed in accordance with embodiments of the present invention, showing the prosthetic sock and the prosthesis cup having layers of force-attenuating elastomeric compound;

FIG. 8 is side view of an endotracheal tube constructed in accordance with embodiments of the present invention, showing the balloon of the endotracheal tube having a force-attenuating elastomeric compound;

FIG. 9 is a perspective view of an intervertebral spacer constructed in accordance with embodiments of the present invention, showing the intervertebral spacer being placed between the endplates of two vertebrae and a hammer having a layer of the force-attenuating elastomeric compound; and

FIG. 10 is a perspective view of a tablet case constructed in accordance with embodiments of the present invention, showing the tablet case having layers of the force-attenuating elastomeric compound.

### DETAILED DESCRIPTION OF THE PREFERRED EMBODIMENTS

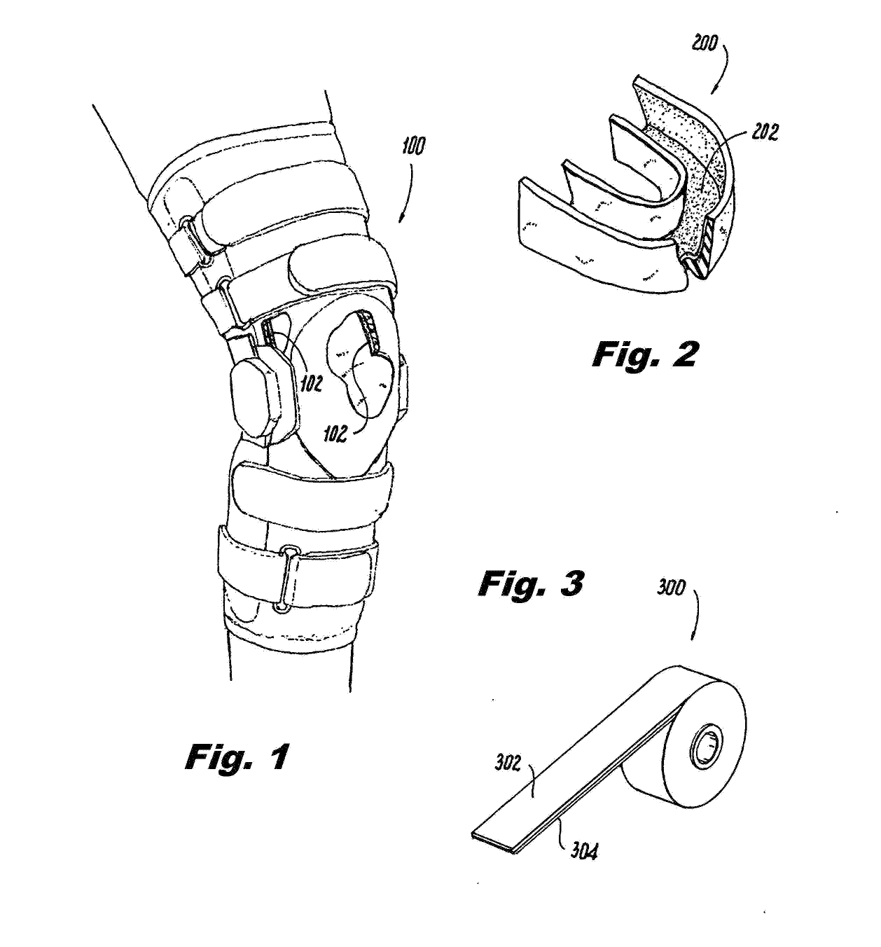

Reference will now be made to the drawings wherein like reference numerals identify similar structural features or aspects of the subject invention. For purposes of explanation and illustration, and not limitation, a perspective view of an exemplary embodiment of a knee brace constructed in accordance with embodiments of the invention is shown in FIG. 1 and is designated generally by reference character 100. Other embodiments in accordance with the invention, or aspects thereof, are provided in FIGS. 2-10, as will be described.

As shown in FIG. 1, a knee brace 100 includes a layer 102 of a polymeric compound within the brace. Specifically, a layer 102 of a viscoelastic, force-attenuating, elastomeric compound, referred to as “the gel” or “gel” throughout this description. It is contemplated that the gel can be the gel described in U.S. Pat. No. 7,041,719, the contents of which are hereby incorporated in their entirety. The gel is placed proximate to bony surfaces such as such as the tibial tubercle and patella, as well as laterally along the femoral condyles. Those skilled in the art will readily appreciate that the gel can be integrated into to a stocking including underneath the hard brace. While only knee brace 100 is shown, a variety of braces can include layers of the gel, for example, back, or elbow braces, namely those for used active sport applications such as football, to reduce impact stresses applied from the structural component of the brace to the limb. Layers 102 act to absorb stress due to dynamic impact to brace 100 and additionally act to disperse static forces, such as continuous compression from brace 100 acting on the user over time.

With reference now to FIG. 2, a mouth guard 200 includes a layer 202 of a gel, similar to the gel described above, to prevent and/or reduce injury to the teeth, for example, during grinding of molars during sleep or during athletic activities. Given the soft nature of the gel, mouth guard 200 may be more comfortable and safer than traditional mouth guards. Layer 202 acts to absorb stress due to dynamic impact to mouth guard 200, and additionally acts to disperse static forces, such as continuous compression from mouth guard 200 acting on the user over time, or teeth clenching by the user.

As shown in FIG. 3, a roll of tape 300 includes a layer 302 of gel similar to the gel describe above and an adhesive 304 applied to one surface. It is contemplated that tape 300 can be applied to specific body parts for protection. This can include knees, elbows and/or any site of previous injury.

It is also contemplated that sports gear for sports such as skiing can have layers of the gel applied to areas such as the elbows of jackets or shirts. The gel can be applied to the hip areas of the insulated pants. In specific sports such as skiing, ice skating, hockey, roller blading, and bicycling, the inclusion of gel layers in the seat of shorts or pants can act to reduce impact over the ischial tuberosities. In addition to using layers of the gel as padding, the gel material can be bonded directly to, or woven into the fabric fibers, for example, they can be infused within the fabric itself and provide improved force and impact attenuation.

In accordance with yet another embodiment, a layer of the gel can be incorporated into bicyclist stockings, or the gel material can be infused directly into the stocking fabric. The medial malleolus can be injured when cyclists attempt quick mounting and dismounting of bicycles. This layer of gel or the gel infused fabric can protect the medial malleolus of the wearer. It is also contemplated that a layer of gel can be incorporated into a bicycle pads and a variety of sports related helmets for enhanced comfort and protection. Moreover, it is contemplated that layers of gel can be incorporated into baseball gloves and batting gloves, or forearm/lower leg/ankle shields to protect against injury due to being hit by the ball. Football padding and helmets can also include layers of gel material.

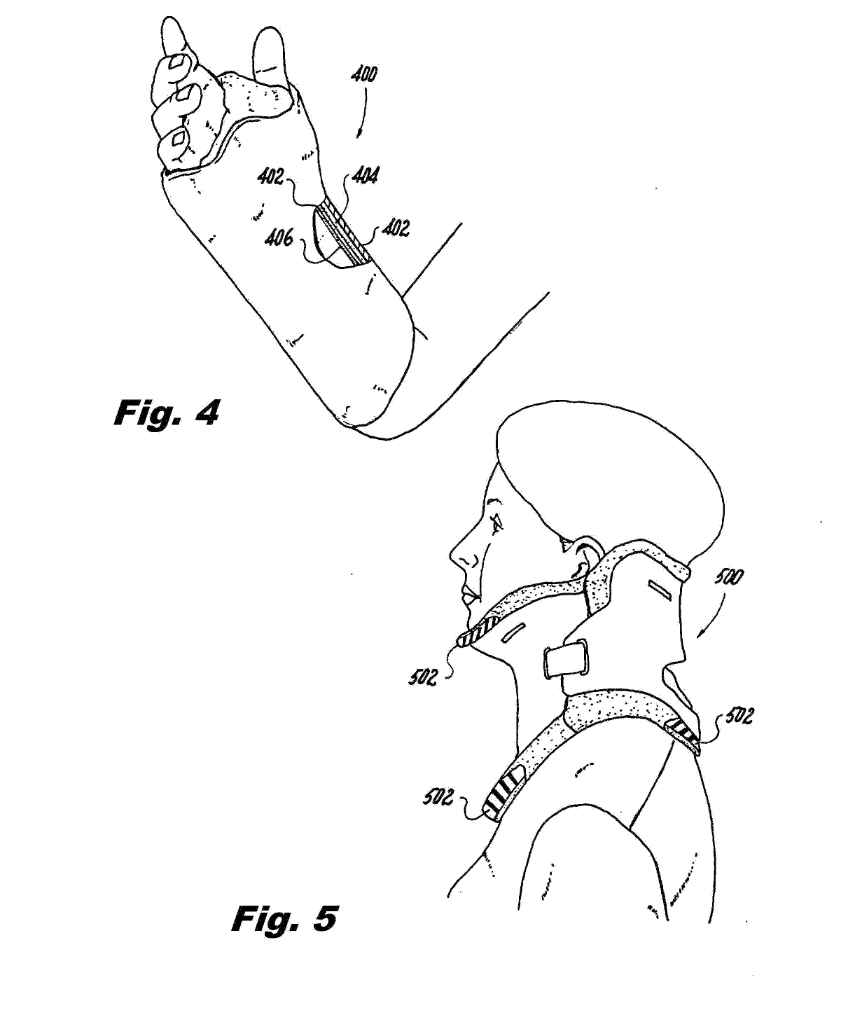

With reference now to FIG. 4, a cast 400 includes layers 402 of a gel, similar to the gel described above, underneath a hard outer cast layer 404 or outside of hard outer cast layer 404. Those skilled in the art will readily appreciate that the hard outer cast layer 404 can be made from plaster of Paris or fiberglass. While the gel is shown underneath and over hard outer cast layer 404, those skilled in the art will readily appreciate that the gel can be incorporated in only one of those locations, or in another suitable location, for example, at the interface between a web-roll layer 406 and the wearer's skin. Layers 402 act to absorb stress due to dynamic impact to cast 400 and additionally act to disperse static forces, such as continuous compression from cast 400 acting on the user over time.

As shown in FIG. 5, a cervical collar 500 includes layers 502 of gel material, similar to the gel described above, incorporated into the padding that surrounds the hard plastic portion of collar 500. When patients have to be immobilized with a traditional cervical collar there tends to be a breakdown of skin related to chafing and pressure necrosis. This can be a complication of using hard collars when immobilizing patients for cervical spine injuries or instability requiring prolonged intubation and sedation when patients cannot communicate pain from contact stress points. In cervical collar 500, layers 502 of gel are applied under the chin (mandibular region) as well as along the posterior occiput and at sternal contact areas to absorb stress due to dynamic impact to collar 500 and additionally acts to disperse stress due to static forces over time such as continuous stress from collar 500 acting on the user over time.

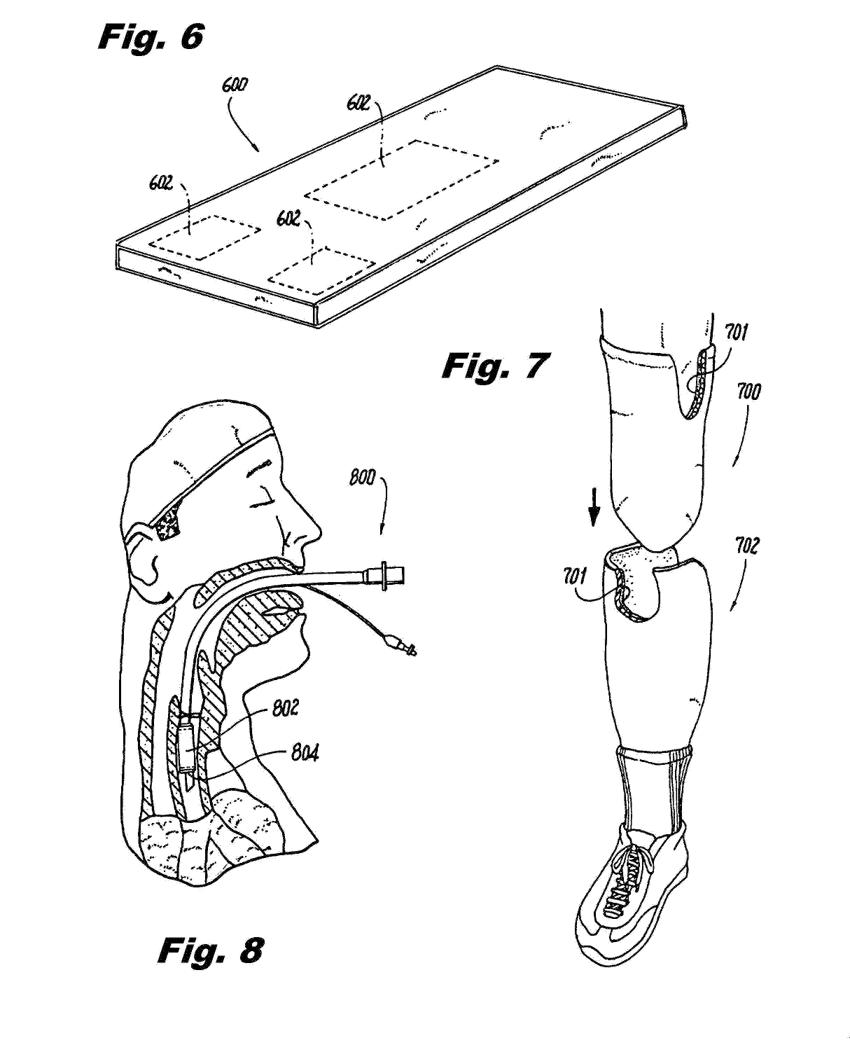

As shown in FIG. 6, a mattress pad 600 includes layers 602 of a gel material, similar to the gel described above, in the sacral region. Mattress pad 600 can be placed under the sheets of a patient's hospital bed to help to reduce and/or prevent sacral decubiti or buttock sores. It is also contemplated that layers 602 of gel material can be incorporated around the heel region to help prevent heel pressure sores. Decubiti are pressure ulcers over the bony prominences in patients who are bed-ridden. It is contemplated that mattress pads 600 can be disposable and/or biodegradable so that they are readily disposable and would have no significant detrimental environmental effect. Conventional methods to prevent decubiti include using air mattresses which are extremely expensive and cumbersome. Layers 602 of gel material acts to disperse stress due to static forces over time, such as the continuous stress from a patient lying on a hospital bed for a pro-longed period of time. Layers 602 of gel material can be less expensive and easier to work with than traditional air mattresses.

In accordance with another embodiment, a shoe insole includes layers of gel material, similar to the gel described above. The layers of gel material are arranged around areas of skin necrosis generally experienced by those with diabetic skin ulcers. It is contemplated that socks can be made with the sole having the gel material included so that the sole can be used with any type of shoe. Socks or heal-cups can also include gel layers to prevent heal breakdown. The gel material can also be directly incorporated into the shoe material, e.g. infused into the shoe material. Specific boots incorporating the gel may be beneficial for athletic or military purposes, or for prevention of plantar fasciitis.

With reference now to FIG. 7, limb sock 700 includes a layer 701 of a gel, similar to the gel described above, to inhibit tissue breakdown where there is direct contact with the prosthesis cup 702 at the end of the amputated limb. Prosthesis cup 702 also includes a layer 701 of the gel. With traditional limb socks and prosthesis cups, continual stress on the end of the amputated limb can cause skin breakdown, not only for vascular insufficient patients who have wound healing issues, but also for traumatic amputees. Limb socks 700 and prosthesis cups 702 alleviate these issues by absorbing stress due to dynamic impact to the limb and additionally acting to disperse static forces over time, such as continuous pressure from prosthesis cup 702 acting on the end of an amputated limb.

In accordance with another embodiment, a girdle having hip pads can include layers of the gel oriented to protect to the sacrum and the trochanter of the hip. These layers prevent or reduce fractures, such as those sometimes experienced by elderly with osteoporosis and also those experienced by patients recovering post-operatively from hip fractures and total hip replacements. It is also contemplated that the gel material, instead of being in a pad layer, can be infused into the girdle material itself.

As shown in FIG. 8, an endotracheal tube 800 includes a balloon 804 made with a layer 802 of gel, similar to the gel described above. In traditional endotracheal tubes, stress from an inflated endotracheal tube balloon tends to cause chronic pressure on the lining of the larynx and trachea which can result in tracheomalacia. Layer 802 of gel in balloon 804 helps reduce pressure on the lining of the larynx and reduce or prevent such a breakdown by more evenly distributing the continuous static forces acting on the lining of the larynx and trachea. Those skilled in the art will readily appreciate that this might reduce the need for tracheostomy in patients requiring long term intubation on ventilator support.

It is also contemplated that layers of gel material, similar to the gel described above, can be used in wheel chairs on the back and/or seat portions of the chair. These layers can help to reduce and/or prevent sacral decubiti or buttock sores by more evenly distributing the static forces between the user and the chair. It is also contemplated that layers of gel material can be used on the hand rest and/or armpit rest of crutches to more evenly distribute the dynamic forces and the continuous static forces imposed on the user.

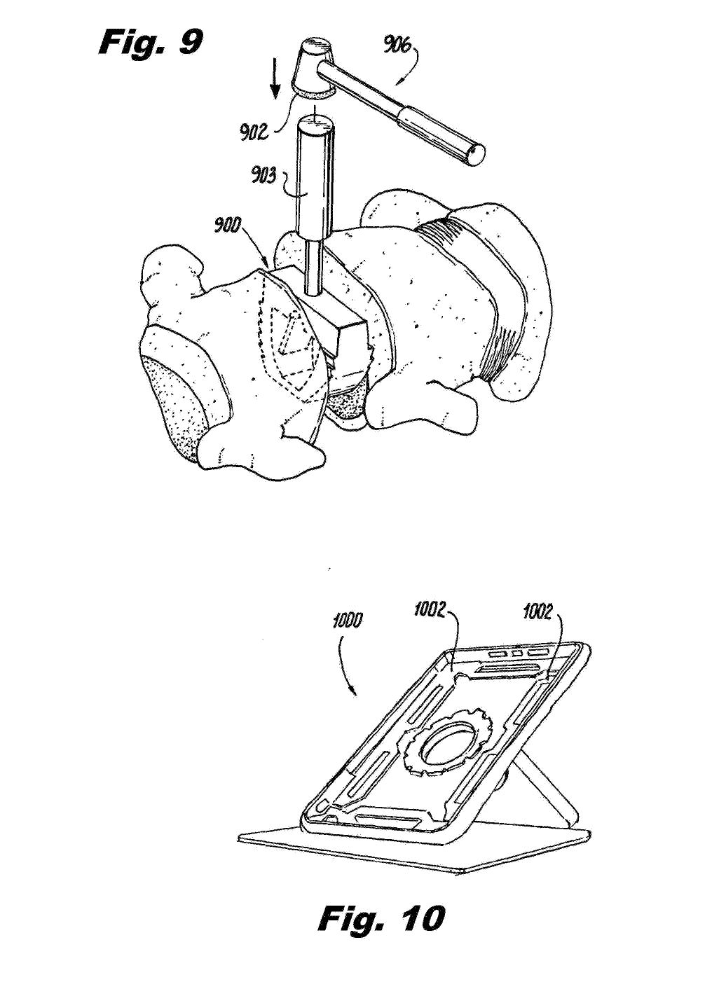

As shown in FIG. 9, an intervertebral implant 900 is being inserted and hammered between vertebrae. A hammer 906 includes a layer 902 of gel, similar to the gel described above, on a distal facing side to protect implant 900 from impact damage while being hammered in. Hammer 906 interfaces with a placement and insertion tool 903 to evenly transmit force to implant 900. Layer 902 of gel can also be a separate sheet placed in situ during the procedure between hammer 906 and tool 903, or between tool 903 and implant 900, but is then removed.

Implant 900 has been described as an exemplary application of an orthopedic implant in accordance with the subject invention. Those skilled in the art will readily appreciate that any other suitable orthopedic application can attain the advantages described herein without departing from the spirit and scope of the invention. For example, the implant can be an intervertebral spacer, an acetabular cup, a glenoid fossa prosthesis, scaphoid prosthesis, or any other suitable type of implant, and the implant location can be in a cervical spine, thoracic spine, lumbar spine, glenohumoral joint, hip joint, wrist, or in any other suitable location. The systems and methods of the invention reduce the amount of trauma during implantation, allowing for faster recovery time.

With reference now to FIG. 10, a tablet case 1000 includes layers 1002 of gel on an inner surface to interface with the tablet surface. Gel layers 1002 are oriented around the perimeter of tablet case 1000 to protect against impact from general use, accidental dropping, and the like. It is also contemplated that gel layers 1002 can be incorporated into a variety of cases to protect computers and other instruments (radios, night vision goggles, etc.) taken into battle zones by soldiers, or used in other rugged environments.

It is also contemplated that a layer of gel can be applied as a durable film over the body of a car or other vehicle, etc., for example in the military/police context, to protect against gunfire, explosions, and accidents etc. It can also be used on civilian vehicles for accident protection.

The gel material also has adhesive qualities that can be used in a variety of applications. For example, the gel material can be applied to the shower or bath tub areas and used as a bath mat to provide surface friction to prevent slips or falls. Additionally, the gel material can be used to prevent injury during a slip or fall, e.g. by attenuating the impact forces during the fall. The gel material can be used as an entry way mat to remove dirt from the soles of shoes. For example, when stepping on the entry mat the adhesive properties of the gel material will collect the dirt from the soles of shoes and reduce the amount of dirt going into the home, business, etc. It is contemplated that the entry mats can be disposable, but also can be re-used after simple washing.

Layers of the gel material can also be used in cases, or organization boxes to maintain position of the components placed in the case. For example, in fishing cases, the gel layer can maintain alignment of lures, hooks, etc. Additionally, for medical applications and other applications that require shipment of sensitive items, the gem material can keep items, such as surgical implants, in a stable fashion when shipping in crates. The gel material can also be used for position maintenance of devices, such as tools during surgery.

The methods and systems of the present invention, as described above and shown in the drawings, provide for medical devices, tools, and electronics cases with superior properties including improved static force distribution and impact attenuation. While the apparatus and methods of the subject invention have been shown and described with reference to preferred embodiments, those skilled in the art will readily appreciate that changes and/or modifications may be made thereto without departing from the spirit and scope of the subject invention.

What is claimed is:

1. A device with a layer of a viscoelastic, force-attenuating, elastomeric compound as recited in the description.

2. An orthopedic implant system comprising:
an implant configured and adapted for implantation in orthopedic applications;
an insertion tool operatively connected to the implant during insertion of the implant; and
a hammer having a layer of gel on a distal facing side configured to engage with the insertion tool during insertion of the implant.
